(19) United States
(12) Patent Application Publication
(10) Pub. No.: US 2010/0093751 A1
(43) Pub. Date: Apr. 15, 2010

(54) Indolizineacetic Acids and Their Therapeutic Use as Ligands of the CRTH2 Receptor

(76) Inventors: George Hynd, Essex (GB); John Gary Montana, Essex (GB); Harry Finch, Essex (GB); Trevor Harrison, Essex (GB); Janusz Kulagowski, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/520,004

(22) PCT Filed: Dec. 13, 2007
(86) PCT No.: PCT/GB2007/004789

§ 371 Date: Aug. 5, 2009

(30) Foreign Application Priority Data
Dec. 22, 2006 (GB) 0625842.0

(51) Int. Cl. A61K 31/437; C07D 471/04; A61K 31/496; A61K 31/506; A61P 11/00; A61P 11/06; A61P 17/06; A61P 1/00

(52) U.S. Cl. 514/253.04; 546/112; 544/333; 544/362; 514/299; 514/256

(57) ABSTRACT

Compounds of formula (I) are CRTH2 ligands, useful in the treatment of, inter alia, respiratory diseases: wherein R¹ is fluoro, chloro, cyano or trifluoromethyl; R² is hydrogen, fluoro or chloro; R³ is hydrogen, fluoro, chloro or trifluoromethyl; X is —CH₂—, —S—, —S(═O)— or —S(═O)₂—; one of Y and Y¹ is hydrogen and the other is —C(═O)R⁴, or —S(═O)₂R⁴, or —CR⁵R⁶OR⁷ or a heterocyclic group selected from furanyl, thienyl, pyrrolyl, oxazolyl, thiazolyl, imidazolyl, pyrazolyl, isoxazolyl, isothiazolyl, 1,2,3-oxadiazolyl, 1,2,4-oxadiazolyl, 1,3,4-oxadiazolyl, 1,2,5-oxadiazolyl, furazanyl, 1,2,3-triazolyl, 1,2,4-triazolyl, 1,2,3-thiadiazolyl, 1,2,5-thiadiazolyl, 1,3,4-thiadiazolyl, 1,2,4-thiadiazolyl, tetrazolyl, pyridinyl, pyridazinyl, pyrimidinyl, pyrazinyl, 1,2,4-triazinyl and 1,3,5-triazinyl any of which may be optionally substituted; R⁴ is an optionally substituted cyclic amino group having 5, 6 or 7 ring atoms which is linked to the carbonyl or sulfonyl through a ring nitrogen; R⁵ and R⁶ are independently hydrogen, (C₁C₃)alkyl, cyclopropyl, or R⁵ and R⁶ taken together with the carbon atom to which they are attached form a 3-6 membered cycloalkyl ring; and R⁷ is optionally substituted (C₁-C₆)alkyl or (C₃-C₆)cycloalkyl,

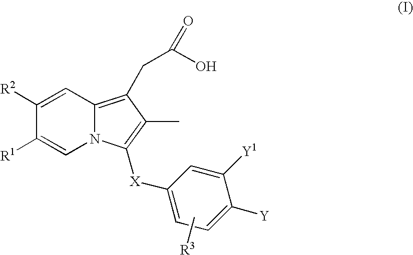

### FIELD OF THE INVENTION

This invention relates to a class of indolizine compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component. The invention also relates to novel members of that class of ligands and pharmaceutical compositions containing them.

### BACKGROUND OF THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major metabolite produced by the action of cyclooxygenase on arachadonic acid by mast cells in response to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al; Arch. Otolarynol. Head Neck Surg., 1987, 113, 179-183; Lebel et al; J. Allergy Clin. Immunol., 1988, 82, 869-877), bronchial asthma (Murray et al; N. Engl. J. Med., 1986, 315, 800-804; Liu et al; Am. Rev. Respir. Dis., 1990, 142, 126-132; Wenzel et al; J. Allergy Clin. Immunol., 1991, 87, 540-548), and urticaria (Heavey et al; J. Allergy Clin. Immunol., 1986, 78, 458-461). PGD₂ mediates it effects through two receptors, the PGD₂ (or DP) receptor (Boie et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonise the effects of PGD₂ at its receptors may have beneficial effects in number of disease states.

The CRTH2 receptor has been shown to be expressed on cell types associated with allergic inflammation, such as basophils, eosinophils, and Th2-type immune helper cells (Hirai et al; J. Exp. Med., 2001, 193, 255-261). The CRTH2 receptor has been shown to mediate PGD₂-mediated cell migration in these cell types (Hirai et al; J. Exp. Med., 2001, 193, 255-261), and also to play a major role in neutrophil and eosinophil cell recruitment in a model of contact dermatitis (Takeshita et al; Int. Immunol., 2004, 16, 947-959). Ramatroban {(3R)-3-[(4-fluorophenyl)-sulphonylamino]-1,2,3,4-tetrahydro-9H-carbazole-9-propanoic acid}, a dual CRTH2 and thromboxane A₂ receptor antagonist, has been shown to attenuate these responses (Sugimoto et al; J. Pharmacol. Exp. Ther., 2003, 305, 347-352; Takeshita et al; op. cit.). The potential of PGD₂ both to enhance allergic inflammation and induce an inflammatory response has been demonstrated in mice and rats. Transgenic mice over expressing PGD₂ synthase exhibit an enhanced pulmonary eosinophilia and increased levels of Th2 cytokines in response to allergen challenge (Fujitani et al, J. Immunol., 2002, 168, 443-449). In addition, exogenously administered CRTH2 agonists enhance the allergic response in sensitised mice (Spik et al; J. Immunol., 2005, 174, 3703-3708). In rats exogenously applied CRTH2 agonists cause a pulmonary eosinophilia but a DP agonist (BW 245C) or a TP agonist (I-BOP) showed no effect (Shirashi et al; J. Pharmacol. Exp Ther., 2005, 312, 954-960). These observations suggest that CRTH2 antagonists may have valuable properties for the treatment of diseases mediated by PGD₂.

In addition to ramatroban a number of other CRTH2 antagonists have been described. Examples include: indole-acetic acids (WO2003/022813; WO2003/066046; WO2003/066047; WO2003/097042; WO2003/097598; WO2003/101961; WO2003/101981; WO2004/007451; WO2004/078719; WO2004/106302; WO2005/019171; GB2407318; WO2005/040112; WO2005/040114; WO2005/044260); tetrahydroquinolines (EP1413306; EP1435356; WO2004/032848; WO2004/035543; WO2005/007094), phenylacetic acids (WO2004/058164; WO2004/089884; WO2004/089885; WO2005/018529) and indolizine acetic acids (WO 2007/031747 and WO 2006/136859).

### DETAILED DESCRIPTION OF THE INVENTION

According to the present invention, there is provided a compound of formula (I) or a salt, N-oxide, hydrate, or solvate thereof:

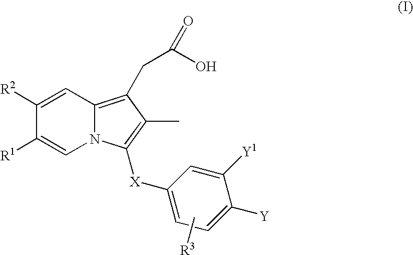

wherein
R¹ is fluoro, chloro, cyano or trifluoromethyl;
R² is hydrogen, fluoro or chloro;
R³ is hydrogen, fluoro, chloro or trifluoromethyl;
X is —CH₂—, —S—, —S(═O)— or —S(═O)₂—;
one of Y and Y¹ is hydrogen and the other is —C(═O)R⁴, or —S(═O)₂R⁴, or —CR⁵R⁶OR⁷ or a heterocyclic group selected from furanyl, thienyl, pyrrolyl, oxazolyl, thiazolyl, imidazolyl, pyrazolyl, isoxazolyl, isothiazolyl, 1,2,3-oxadiazolyl, 1,2,4-oxadiazolyl, 1,3,4-oxadiazolyl, 1,2,5-oxadiazolyl, furazanyl, 1,2,3-triazolyl, 1,2,4-triazolyl, 1,2,3-thiadiazolyl, 1,2,5-thiadiazolyl, 1,3,4-thiadiazolyl, 1,2,4-thiadiazolyl, tetrazolyl, pyridinyl, pyridazinyl, pyrimidinyl, pyrazinyl, 1,2,4-triazinyl and 1,3,5-triazinyl any of which may be optionally substituted;

R⁴ is an optionally substituted cyclic amino group having 5, 6 or 7 ring atoms which is linked to the carbonyl or sulfonyl through a ring nitrogen;

R⁵ and R⁶ are independently hydrogen, (C₁-C₃)alkyl, cyclopropyl, or R⁵ and R⁶ taken together with the carbon atom to which they are attached form a 3-6 membered cycloalkyl ring; and

R⁷ is optionally substituted (C₁-C₆)alkyl or (C₃-C₆)cycloalkyl.

Compounds (I) with which the invention is concerned are CRTH2 receptor antagonists, but they may also have beneficial effects at other prostanoid receptors, such as the PGD₂ receptor or the thromboxane A₂ receptor.

A second aspect of the invention is a pharmaceutical composition comprising a compound of formula (I), or a salt, N-oxide, hydrate or solvate thereof, in admixture with a pharmaceutically acceptable carrier or excipient.

A third aspect of the invention is a compound of formula (I), or a salt, N-oxide, hydrate or solvate thereof, for use in therapy.

A fourth aspect of the invention is the use of a compound of formula (I), or a salt, N-oxide, hydrate or solvate thereof, in the manufacture of a medicament for the treatment of a disease in which a CRTH2 antagonist can prevent, inhibit or ameliorate the pathology and/or symptomatology of the disease.

A fifth aspect of the invention is a method for treating a disease in a patient in which a CRTH2 antagonist can prevent, inhibit or ameliorate the pathology and/or symptomatology of the disease, which method comprises administering to the patient a therapeutically effective amount of compound of formula (I), or a salt, N-oxide, hydrate or solvate thereof,

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment of asthma, chronic obstructive pulmonary disease, rhinitis, allergic airway syndrome, and allergic rhinobronchitis; and also for the treatment of psoriasis, atopic and non-atopic dermatitis, Crohn's disease, ulcerative colitis, and irritable bowel disease.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “fully or partially fluorinated Cₐ-C_{b}alkyl” wherein a and b are integers refers to a straight or branched chain alkyl radical having from a to b carbon atoms in which the hydrogen atoms all replaced by fluorine (fully fluorinated) or in which some of the hydrogen atoms are replaced by fluorine (partially fluorinated). The term includes, for example —CF₃, —CHF₂, —CFH₂, and CF₃CH₂—.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “Cₐ-C_{b} alkynyl” wherein a and b are integers refers to straight chain or branched chain hydrocarbon groups having from two to six carbon atoms and having in addition one triple bond. This term would include for example, ethynyl, 1- and 2-propynyl, 1-, 2- and 3-butynyl, 1, 2-, 3- and 4-pentynyl, 1-, 2-, 3-, 4- and 5-hexynyl, 3-methyl-1-butynyl and 1-methyl-2-pentynyl.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, quinolyl, morpholinyl, benzfuranyl, pyranyl, isoxazolyl, benzimidazolyl, methylenedioxyphenyl, ethylenedioxyphenyl, maleimido and succinimido groups.

“Cyclic amino groups” are saturated monocyclic heterocyclic rings of 3 to 8 ring atoms, one of which is nitrogen, but which may also contain other heteroatoms selected from O, N and S. When such cyclic amino groups are covalently linked to another atom, the link is via a ring nitrogen.

Unless otherwise specified in the context in which it occurs, the term “substituted” as applied to any moiety herein means substituted with up to four compatible substituents, each of which independently may be, for example, (C₁-C₆)alkyl, cycloalkyl, (C₁-C₆)alkoxy, hydroxy, hydroxy(C₁-C₆)alkyl, mercapto, mercapto(C₁-C₆)alkyl, (C₁-C₆)alkylthio, phenyl, monocyclic heteroaryl having 5 or 6 ring atoms, halo (including fluoro, bromo and chloro), trifluoromethyl, trifluoromethoxy, nitro, nitrile (—CN), oxo, —COON, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NR^{A}COR^{B}; —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl , phenyl or monocyclic heteroaryl having 5 or 6 ring atoms, or R^{A} and R^{B} when attached to the same nitrogen atom form a cyclic amino ring, such as piperidinyl, morpholinyl or piperazinyl. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)aminomethane, L-arginine, L-lysine, N-ethyl piperidine, dibenzylamine and the like. Specific salts with bases include the benzathine, calcium, diolamine, meglumine, olamine, potassium, procaine, sodium, tromethamine and zinc salts. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

Use of prodrugs, such as esters, of compounds (I) with which the invention is concerned is also part of the invention. “Prodrug” means a compound which is convertible in vivo by metabolic means (e.g. by hydrolysis, reduction or oxidation) to a compound of formula (I). For example an ester prodrug of a compound of formula (I) may be convertible by hydrolysis in vivo to the parent molecule. Suitable esters of compounds of formula (I) are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluene-sulphonates, cyclohexylsulphamates and quinates. Examples of ester prodrugs are those described by F. J. Leinweber, Drug Metab. Res., 1987, 18, 379. As used in herein, references to the compounds of formula (I) are meant to also include the prodrug forms.

In the compounds of the invention, the following structural features may be present, in any compatible combination:
Of the allowed possibilities for X, it is currently preferred that X be —CH₂— or —S—.
Optional substituents in Y or Y¹ will generally be small hydrophobic substituents, and may be selected from, for example, halogen such as fluoro, chloro and bromo, —CN, C₁-C₃ alkyl such as methyl, fully or partially fluorinated C₁-C₃alkyl such as trifluoromethyl, and cyclopropyl.
When Y or Y¹ is —C(═O)R⁴, or —S(═O)₂R⁴, R⁴ may be, for example, morpholinyl, piperidinyl, piperizanyl, N-substituted piperazinyl such as 4-methylpiperazinyl, or pyrrolidinyl. Currently it is preferred than Y or Y¹ be —S(═O)₂R⁴.
Of the allowed possibilities when Y or Y¹ is a heterocyclic group, oxazolyl, pyrazolyl, pyridinyl, pyrimidinyl, are specific examples.
In many embodiments of the invention, Y¹ will be hydrogen.
When Y or Y¹ is —CR⁵R⁶OR⁷, R⁵ and and R⁶ may be, for example, independently hydrogen or methyl or cyclopropyl, and R⁷ may be for example methyl or ethyl.

Specific examples of compounds with which the invention is concerned include those of the examples herein.

### Compositions

As mentioned above, the compounds with which the invention is concerned are CRTH2 receptor antagonists, and are useful in the treatment of diseases which benefit from such modulation. Examples of such diseases are referred to above, and include asthma, rhinitis, allergic airway syndrome, allergic rhinobronchitis and chronic obstructive pulmonary disease.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range will lie within the range of from about 0.001 mg to about 100 mg per kg body weight of a mammal, often 0.01 mg to about 50 mg per kg, for example 0.1 to 10 mg per kg, in single or divided doses. On the other hand, it may be necessary to use dosages outside these limits in some cases.

The compounds with which the invention is concerned may be prepared for administration by any route consistent with their pharmacokinetic properties. Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers. For delivery by inhalation, the active compound is preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

Other compounds may be combined with compounds of this invention of formula (I) for the prevention and treatment of prostaglandin-mediated diseases. Thus the present invention is also concerned with pharmaceutical compositions for preventing and treating PGD₂-mediated diseases comprising a therapeutically effective amount of a compound of the invention of formula (I) and one or more other therapeutic agents. Suitable therapeutic agents for a combination therapy with compounds of formula (I) include, but are not limited to: (1) corticosteroids, such as fluticasone, budesonide or ciclesonide; (2) β2-adrenoreceptor agonists, such as salmeterol, formeterol or indacaterol; (3) leukotriene modulators, for example leukotriene antagonists such as montelukast or pranlukast or leukotriene biosynthesis inhibitors such as Zileuton or BAY-x1005; (4) anticholinergic agents, for example muscarinic-3 (M₃) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TNF-α inhibitors, for example anti-TNF monoclonal antibodies, such as Remicade and CDP-870 and TNF receptor immunoglobulin molecules, such as Enbrel; (12) inhibitors of matrix metalloprotease (MMP), for example MMP8, 9 and 12; (13) human neutrophil elastase inhibitors, such as those described in WO2005/026124 and WO2003/053930; (14) Adenosine A2a agonists such as those described in EP1052264 and EP1241176 (15) Adenosine A2b antagonists such as those described in WO2002/42298; (16) modulators of chemokine receptor function, for example antagonists of CCR3 and CCR8; (17) compounds which modulate the action of other prostanoid receptors, for example a PGD2 (DP) receptor antagonist or a thromboxane A2 antagonist; and (18) compounds which modulate Th2 function, for example, PPAR agonists.

The weight ratio of the compound of the invention to the second active ingredient may be varied and will depend upon the effective dose of each ingredient. Generally, an effective dose of each will be used.

### Methods of Synthesis

The present invention is also concerned with processes for preparing the compounds of this invention.

The compounds of formula (I) of the present invention can be prepared according to the procedures of the following schemes and examples, using appropriate materials, and are further exemplified by the following specific examples. Moreover, by utilizing the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention of formula (I) may be isolated in the form of their pharmaceutically acceptable salts, such as those described previously herein. The free acid form corresponding to isolated salts can be generated by acidification with a suitable acid such as acetic acid and hydrochloric acid and extraction of the liberated free acid into an organic solvent followed by evaporation. The free acid form isolated in this manner can be further converted into another pharmaceutically acceptable salt by dissolution in an organic solvent followed by addition of the appropriate base and subsequent evaporation, precipitation, or crystallisation.

It may be necessary to protect reactive functional groups (e.g. hydroxy, amino, thio or carboxy) in intermediates used in the preparation of compounds of formula (I) to avoid their unwanted participation in a reaction leading to the formation of compounds of formula (I). Conventional protecting groups, for example those described by T. W. Greene and P. G. M. Wuts in “Protective groups in organic chemistry” John Wiley and Sons, 1999, may be used.

Compounds of formula (I) wherein X is a —S— group are represented by compounds of formula (I-b). Compounds of formula (I-b) wherein R^{a} is as defined for Y and R^{b} is as defined for Y¹ in formula (I) may be prepared according to the route outlined in Scheme 1.

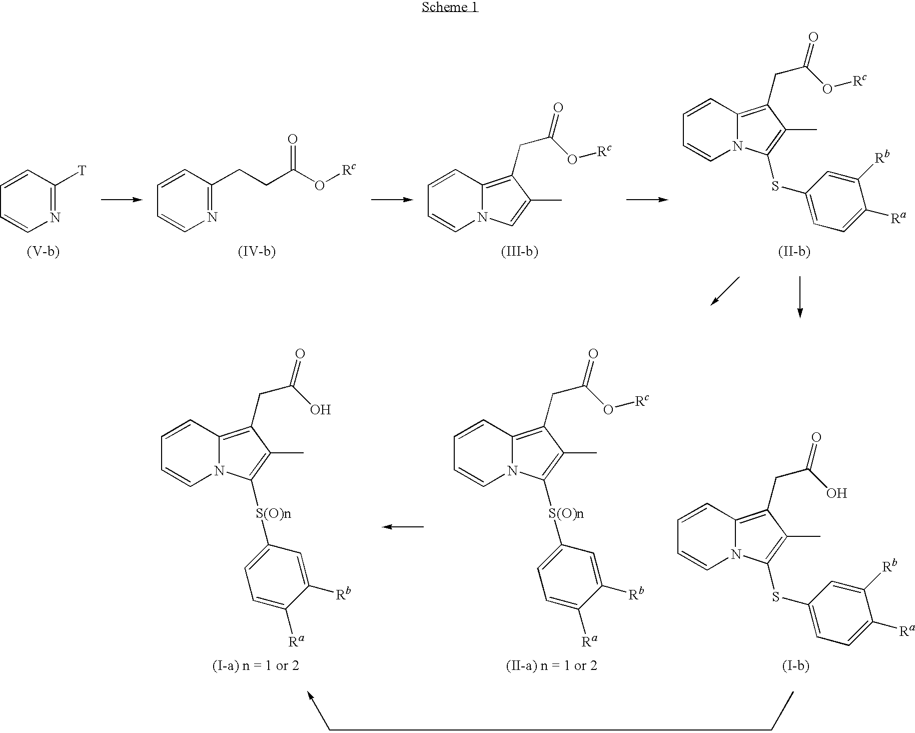

Compounds of formula (I-b) may be prepared from compounds of formula (II-b) wherein R^{a} and R^{b} are as defined above and R^{c} is a lower alkyl group, by hydrolysis of the ester group under standard conditions familiar to those skilled in the art. For example, by treatment with a metal hydroxide such as lithium hydroxide in a polar protic solvent such as an alcohol, preferably methanol, in the presence of water. The reaction may be conducted at a temperature between 0° C. and the reflux temperature of the solvent, preferably at ambient temperature.

Compounds of formula (II-b) may be prepared from compounds of formula (III-b) wherein R^{c} is as defined above, by treatment with a disulfide of formula (VI);

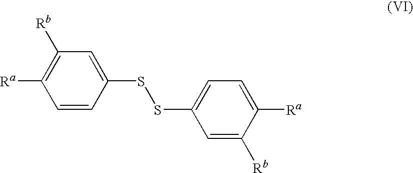

wherein R^{a} and R^{b} are as described above. The reaction takes place in the presence of sulfuryl chloride in a suitable solvent such as dichloromethane or dichloroethane, at a temperature between 0° C. and the reflux temperature of the solvent, preferably at ambient temperature. Compounds of formula (VI) are commercially available or are known compounds or can readily be prepared from known compounds using methods described in the literature.

Compounds of formula (III-b) may conveniently be prepared by the reaction between a compound of formula (IV-b) and a suitable alkylating agent of formula (VII), wherein LG represents a suitable leaving group such as chloro, bromo, or methanesulfonyloxy. Typically, the alkylation reaction is carried out in the presence of a base such as sodium hydrogen carbonate or pyridine in an inert solvent such as acetonitrile.

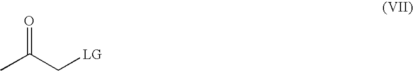

Compounds of formula (IV-b) may be prepared by the reaction between a compound of formula (V-b), in which group T represents a chloro, bromo, or iodo atom, or a trifluoromethanesulfonyloxy group, and a compound of formula (VIII);

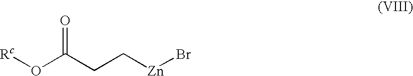

wherein R^{c} is as defined above. The reaction may conveniently be carried out in the presence of a suitable catalyst, such as tetrakis(triphenylphosphine)palladium(0) in an aprotic solvent such as toluene or tetrahydrofuran. Compounds of formula (V-b) and (VIII) are commercially available or prepared by known methods.

Using the route outlined in Scheme 1, compounds of formula (I-a) may be prepared from compounds of formula (II-b) by oxidation/ester hydrolysis via compounds of formula (II-a) or (I-b).

Compounds of formula (I) wherein X is a —CH₂— group are represented by compounds of formula (I-c). Compounds of formula (I-c) wherein R^{a} is as defined for Y and R^{b} is as defined for Y¹ in formula (I) may be prepared according to the route outlined in Scheme 2.

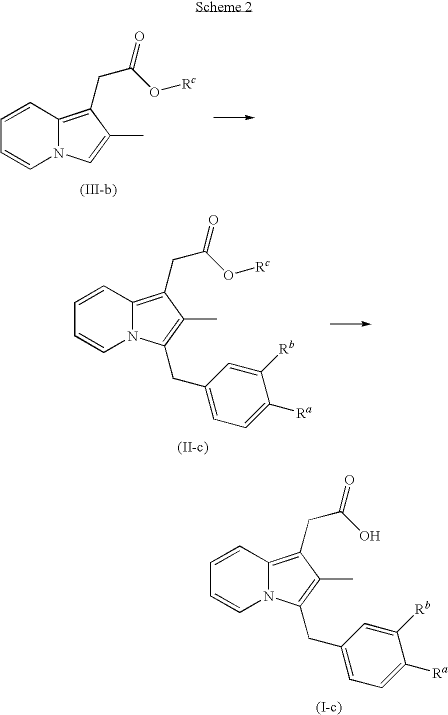

Compounds of formula (I-c) may be prepared from compounds of formula (II-c) wherein R^{a}, R^{b} and R^{c} are as described above, using methods described above for the preparation of compounds of formula (I-b) from compounds of formula (II-b) (Scheme 1). Compounds of formula (II-c) may be conveniently prepared by reaction of compounds of formula (III-b) with compounds of formula (IX);

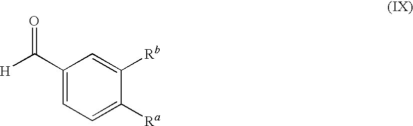

wherein R^{a} and R^{b} are as defined as above, under acidic reductive conditions, for example a mixture of trifluoroacetic acid and triethylsilane. Compounds of formula (IX) are commercially available or can be prepared by methods well known to those skilled in the art.

Alternatively, compounds of formula (I-a), (I-b) and (I-c) wherein R^{a} or R^{b} represents a heterocyclic group may be conveniently prepared from compounds of formula (II-a), (II-b) and (II-c) wherein R^{a} or R^{b} represents chloro, bromo, or iodo atom, or a trifluoromethanesulfonyloxy group, by reaction with a organometallic reagent of formula (X);

M-Het (X)

wherein Het represents a 5- or 6-membered heteroaryl ring and M represents an appropriately substituted boron, zinc, tin or silicon group. The reaction may conveniently be carried out in the presence of a suitable catalyst such as a palladium compound.

### Examples

### Example 1

### {6-fluoro-3-[2-fluoro-4-(morpholine-4-sulfonyl)phenylsulfanyl]-2-methylindolizin-1-yl}acetic acid

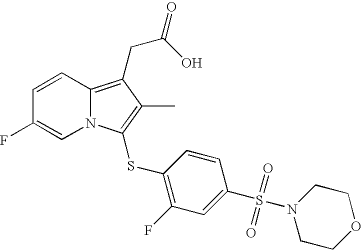

### Preparation 1a: 3-(5-fluoropyridin-2-yl)propionic acid ethyl ester

A solution of 3-ethoxy-3-oxopropylzinc bromide in tetrahydrofuran (0.5 M, 630 mL) was added dropwise to a mixture of 2-bromo-5-fluoropyridine (50 g), tetrakis(triphenylphosphine)palladium(0) (5.5 g) and toluene (350 mL) and the resulting mixture was stirred at room temperature for 24 hours. The mixture was concentrated under reduced pressure, diluted with ethyl acetate and filtered through hyflo. The filtrate was washed with saturated aqueous sodium chloride solution, dried over magnesium sulfate and concentrated under reduced pressure. Purification of the residue by column chromatography on silica gel, eluting with a mixture of pentane and ethyl acetate (9:1 by volume) gave title compound as a yellow oil, 35 g.

¹H NMR (CDCl₃): δ 1.25 (t, J=7.1 Hz, 3H), 2.75 (t, J=7.4 Hz, 2H), 3.10 (t, J=7.4 Hz, 2H), 4.10 (q, J=7.1 Hz, 2H), 7.20 (dd, J=4.4, 8.4 Hz, 1H), 7.30 (dt, J=3.0, 8.4 Hz, 1H), 8.35 (d, J=2.6 Hz, 1H).

### Preparation 1b: (6-fluoro-2-methylindolizin-1-yl)acetic acid ethyl ester

A mixture of 3-(5-fluoropyridin-2-yl)propionic acid ethyl ester (12 g) and acetonitrile (90 mL) at room temperature was treated with a solution of 1-bromopropan-2-one (16 g) in acetonitrile (30 mL) and the resulting mixture was heated at reflux for 24 hours and then left to stand at room temperature for 60 hours. The mixture was treated with pyridine (35 mL) and the resulting mixture was heated at reflux for 4 hours, cooled to room temperature and then concentrated under reduced pressure. The residue was diluted with ethyl acetate, washed with water and saturated aqueous sodium hydrogen chloride solution and then dried over magnesium sulfate. The solvent was removed under reduced pressure and the residue purified by column chromatography on silica gel, eluting with a mixture of pentane and ethyl acetate (19:1 by volume) to afford title compound as a yellow oil, 3.7 g.

¹H NMR (CDCl₃): δ1.25 (t, J=7.1 Hz, 3H), 2.25 (s, 3H), 3.65 (s, 2H), 4.11 (q, J=7.1 Hz, 2H), 6.55 (m, 1H), 7.10 (br s, 1H), 7.25 (m, 1H), 7.70 (d, J=3.4 Hz, 1H).

### Preparation 1c: 4-(3,4-difluorobenzenesulfonyl)morpholine

A solution of 3,4-difluorobenzenesulfonyl chloride (5.0 g) in dichloromethane (20 mL) was added dropwise to a solution of morpholine (6.1 mL) in dichloromethane (30 mL) at 0° C. and the resulting mixture was stirred at 0° C. for 15 minutes and then at room temperature for 20 minutes. The mixture was washed with water, dried over magnesium sulfate and solvent removed under reduced pressure to afford title compound as a white solid, 6.4 g.

¹H NMR (CDCl₃): δ 3.05 (m, 4H), 3.75 (m, 4H), 7.35-7.40 (m, 1H), 7.55 (m, 1H), 7.60 (m, 1H).

### Preparation 1d: bis[2-fluoro-4-(morpholine-4-sulfonyl)benzene]disulfide

A mixture of 4-(3,4-difluorobenzenesulfonyl)morpholine (1.0 g), sodium hydrogensulfide (2.9 g) and 1-methylpyrrolidin-2-one (4.0 mL) was stirred at 80° C. for 90 minutes and then at room temperature for 5 hours. The mixture was diluted with water, washed with ethyl acetate and the aqueous phase acidified by the addition of concentrated hydrochloric acid. The mixture was extracted with ethyl acetate and the combined extracts were dried over magnesium sulfate and then concentrated under reduced pressure. The residue was triturated with water to afford title compound as a white solid, 0.94 g.

MS: ESI (+ve) (Method B): 553 (M+H)⁺, Retention time 3.1 min.

### Preparation 1e: {6-fluoro-3-[2-fluoro-4-(morpholine-4-sulfonyl)phenylsulfanyl]-2-methylindolizin-1-yl}acetic acid ethyl ester

Sulfuryl chloride (0.060 mL) was added to a mixture of bis[2-fluoro-4-(morpholine-4-sulfonyl)benzene]disulfide (0.48 g) and dichloromethane (6.0 mL) at 0° C. and the resulting mixture was stirred at 0° C. for 10 minutes and then at room temperature for 90 minutes. A solution of (6-fluoro-2-methylindolizin-1-yl)acetic acid ethyl ester (0.15 g) in dichloromethane (1.0 mL) was added and the resulting mixture stirred at room temperature for 18 hours. The mixture was washed with saturated aqueous sodium hydrogen carbonate solution, water and saturated aqueous sodium chloride solution and then dried over magnesium sulfate. The solvent was removed under reduced pressure and the residue purified by column chromatography on silica gel, eluting with a mixture of dichloromethane and methanol (1:0 to 99:1 by volume) to afford title compound as a brown solid, 0.53 g.

MS: ESI (+ve) (Method B): 511 (M+H)⁺, Retention time 4.3 min.

### Preparation 1f: {6-fluoro-3-[2-fluoro-4-(morpholine-4-sulfonyl)phenylsulfanyl]-2-methylindolizin-1-yl}acetic acid

A mixture of {6-fluoro-3-[2-fluoro-4-(morpholine-4-sulfonyl)phenylsulfanyl]-2-methylindolizin-1-yl}acetic acid ethyl ester (0.32 g) and methanol (15 mL) was treated with 1.0 M aqueous sodium hydroxide solution (1.5 mL) and the resulting mixture was stirred at room temperature for 3 hours. The mixture was concentrated under reduced pressure, diluted with water and washed with dichloromethane. The aqueous phase was acidified by the addition of glacial acetic acid, extracted with dichloromethane and the combined extracts dried over magnesium sulfate. The solvent was removed under reduced pressure and the residue purified by preparative reverse-phase HPLC using a gradient over 45 minutes of acetonitrile in water (50% to 95% of organic modifier) to afford title compound as a pale green solid, 0.064 g.

¹H NMR (CDCl₃): δ 2.30 (s, 3H), 2.95 (m, 4H), 3.70 (m, 4H), 3.80 (s, 2H), 6.30 (dd, J=7.4, 8.0 Hz, 1H), 6.85 (m, 1H), 7.20 (dd, J=1.7, 8.3 Hz, 1H), 7.40 (dd, J=5.3, 9.6 Hz, 1H), 7.45 (dd, J=1.7, 8.9 Hz, 1H), 8.05 (dd, J=2.1, 4.8 Hz, 1H).

MS: ESI (+ve) (Method A): 483 (M+H)⁺, Retention time 11.1 min.

### Example 2

### {6-fuoro-2-methyl-3-[4-(pyrrolidine-1-carbonyl)phenylsulfanyl]indolizin-1-yl}acetic acid

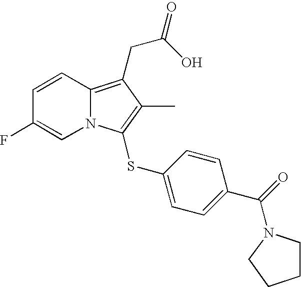

### Preparation 2a

### {4-[4-(pyrrolidine-1-carbonyl)phenyldisulfanyl]phenyl}pyrrolidin-1-ylmethanone

A mixture of bis(4-carboxyphenyl)disulfide (1.5 g), 2-(7-aza-1H-benzotriazole-1-yl)-1,1,3,3-tetramethyluronium hexafluorophosphate (8.2 g), pyrrolidine (2.5 mL), N,N-disopropylethylamine (5.0 mL) and N,N-dimethylformamide (5.0 mL) was stirred at room temperature overnight. The mixture was diluted with ethyl acetate, washed with saturated aqueous sodium hydrogen carbonate solution and water and then dried over sodium sulfate. The solvent was removed under reduced pressure and the residue purified by column chromatography on silica gel, eluting with a mixture of dichloromethane and methanol. Further purification by isolute flash SCX-2 column, eluting with methanol gave title compound, 1.0 g.

MS: ESI (+ve) (Method B): 413 (M+H)⁺, Retention time 3.2 min.

### Preparation 2b

### {6-fluoro-2-methyl-3-[4-(pyrrolidine-1-carbonyl)phenylsulfanyl]indolizin-1-yl}acetic acid ethyl ester

The title compound was prepared by the method of Preparation 1e using (6-fluoro-2-methylindolizin-1-yl)acetic acid ethyl ester and {4-[4-(pyrrolidine-1-carbonyl)phenyldisulfanyl]phenyl}pyrrolidin-1-ylmethanone.

MS: ESI (+ve) (Method B): 441 (M+H)⁺, Retention time 4.0 min.

### Preparation 2c

### {6-fuoro-2-methyl-3-[4-(pyrrolidine-1-carbonyl)phenylsulfanyl]indolizin-1-yl}acetic acid

A solution of {6-fluoro-2-methyl-3-[4-(pyrrolidine-1-carbonyl)phenylsulfanyl]indolizin-1-yl}acetic acid ethyl ester (0.080 g) in tetrahydrofuran (3.0 mL) was treated with a solution of lithium hydroxide (0.038 g) in water (4.0 mL) and the resulting mixture was stirred at room temperature for 2.5 hour. The mixture was acidified by the addition of 1.0 M aqueous hydrochloric acid, extracted with ethyl acetate and the combined extracts concentrated under reduce pressure. The residue was purified by preparative reverse-phase HPLC using a gradient of acetonitrile in water (20% to 98% of organic modifier) to afford title compound as a green solid, 0.024 g.

¹H NMR (CD₃OD): δ 1.80-2.00 (m, 4H), 2.30 (s, 3H), 3.40 (t, J=6.6 Hz, 2H), 3.50 (t, J=6.9 Hz, 2H), 3.75 (s, 2H), 6.80 (m, 1H), 6.85 (d, J=8.5 Hz, 2H), 7.35 (d, J=8.5 Hz, 2H), 7.50 (dd, J=5.2, 9.7 Hz, 1H), 8.05 (dd, J=0.6, 5.2 Hz, 1H).

MS: ESI (+ve) (Method A): 413 (M+H)⁺, Retention time 9.5 min.

### Example 3

### [6-fluoro-2-methyl-3-(4-pyrimidin-5-ylphenylsulfanyl)indolizin-1-yl]acetic acid

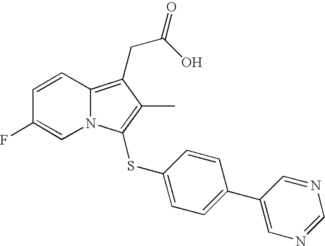

### Preparation 3a

### [3-(4-bromophenylsulfanyl)-6-fluoro-2-methylindolizin-1-yl]acetic acid ethyl ester

The title compound was prepared by the method of Preparation 1e using (6-fluoro-2-methylindolizin-1-yl)acetic acid ethyl ester and 4-bromobenzenethiol.

MS: ESI (+ve) (Method B): Retention time 4.9 min.

### Preparation 3b

### [6-fluoro-2-methyl-3-(4-pyrimidin-5-ylphenylsulfanyl)indolizin-1-yl]acetic acid ethyl ester

A mixture of [3-(4-bromophenylsulfanyl)-6-fluoro-2-methylindolizin-1-yl]acetic acid ethyl ester (0.20 g), pyrimidine-5-boronic acid (0.087 g) palladium (0) tetrakis(triphenylphosphine) (0.027 g), 2.0 M cesium carbonate (1.0 mL) and N,N-dimethylformamide (4.0 mL) was heated by microwave irradiation at 100° C. for 60 seconds. The mixture was concentrated under reduced pressure, diluted with dichloromethane, washed with water and then dried over magnesium sulfate. The solvent was removed under reduced pressure and the residue used in the next step without further purification.

MS: ESI (+ve) (Method B): 422 (M+H)⁺, Retention time 4.1 min.

### Preparation 3c

### [6-fluoro-2-methyl-3-(4-pyrimidin-5-ylphenylsulfanyl)indolizin-1-yl]acetic acid

A mixture of [6-fluoro-2-methyl-3-(4-pyrimidin-5-ylphenylsulfanyl)indolizin-1-yl]acetic acid ethyl ester (0.20 g) and methanol (5.0 mL) was treated with 5.0 M aqueous sodium hydroxide solution (1.0 mL) and the resulting mixture was stirred at room temperature overnight. The mixture was concentrated under reduced pressure, acidified by the addition of glacial acetic acid and then purified by preparative reverse-phase HPLC using a gradient over 30 minutes of acetonitrile in water (50% to 95% of organic modifier) to afford title compound as a pale yellow solid, 0.0075 g.

¹H NMR (DMSO-d6): δ2.25 (s, 3H), 3.70 (s, 2H), 6.95 (m, 3H), 7.60 (m, 1H), 7.65 (d, J=8.6 Hz, 2H), 8.20 (dd, J=2.1, 5.1 Hz, 1H), 9.00 (s, 2H), 9.10 (s, 1H).

MS: ESI (+ve) (Method A): 394 (M+H)⁺, Retention time 10.4 min.

MS: ESI (+ve) (Method B): 394 (M+H)⁺, Retention time 3.4 min.

### Example 4

### {6-fluoro-2-methyl-3-[4-(1-methyl-1H-pyrazol-4-yl)phenylsulfanyl]indolizin-1-yl}acetic acid

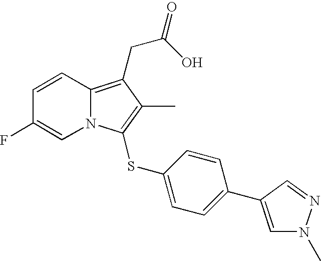

### Preparation 4a

### {6-fluoro-2-methyl-3-[4-(1-methyl-1H-pyrazol-4-yl)phenylsulfanyl]indolizin-1-yl}acetic acid ethyl ester

The title compound was prepared by the method of Preparation 3b using [3-(4-bromophenylsulfanyl)-6-fluoro-2-methylindolizin-1-yl]acetic acid ethyl ester and 1-methylpyrazole-4-boronic acid pinacol ester.

MS: ESI (+ve) (Method B): 424 (M+H)⁺, Retention time 4.2 min.

### Preparation 4b

### {6-fluoro-2-methyl-3-[4-(1-methyl-1H-pyrazol-4-yl)phenylsulfanyl]indolizin-1-yl}acetic acid

The title compound was prepared by the method of Preparation 3c using {6-fluoro-2-methyl-3-[4-(1-methyl-1H-pyrazol-4-yl)phenylsulfanyl]indolizin-1-yl}acetic acid ethyl ester.

¹H NMR (DMSO-d6): δ 2.25 (s, 3H), 3.70 (s, 2H), 3.75 (s, 3H), 6.85 (d, J=8.4 Hz, 2H), 6.90 (m, 1H), 7.40 (d, J=5.7, 9.7 Hz, 2H), 7.60 (dd, J=5.7, 9.7 Hz, 1H), 7.70 (s, 1H), 8.00 (s, 1H), 8.20 (dd, J=2.1, 5.2 Hz, 1H).

MS: ESI (+ve) (Method A): 396 (M+H)⁺, Retention time 10.8 min.

MS: ESI (+ve) (Method B): 396 (M+H)⁺, Retention time 3.6 min.

### Example 5

### [6-fluoro-2-methyl-3-(4-pyridin-2-ylphenylsulfanyl)indolizin-1-yl]acetic acid

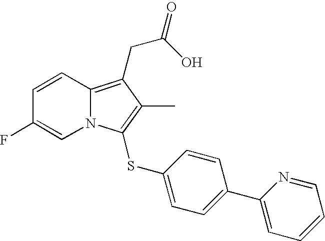

### Preparation 5a

### [6-fluoro-2-methyl-3-(4-pyridin-2-ylphenylsulfanyl)indolizin-1-yl]acetic acid ethyl ester

The title compound was prepared by the method of Preparation 3b using [3-(4-bromophenylsulfanyl)-6-fluoro-2-methylindolizin-1-yl]acetic acid ethyl ester and pyridine-2-boronic acid dimethyl ester.

MS: ESI (+ve) (Method B): 421 (M+H)⁺, Retention time 4.2 min.

### Preparation 5b

### [6-fluoro-2-methyl-3-(4-pyridin-2-ylphenylsulfanyl)indolizin-1-yl]acetic acid

The title compound was prepared by the method of Preparation 3c using [6-fluoro-2-methyl-3-(4-pyridin-2-ylphenylsulfanyl)indolizin-1-yl]acetic acid ethyl ester.

MS: ESI (+ve) (Method A): 393 (M+H)⁺, Retention time 10.0 min.

### Example 6

### [6-fluoro-2-methyl-3-(4-oxazol-2-ylphenylsulfanyl)indolizin-1-yl]acetic acid

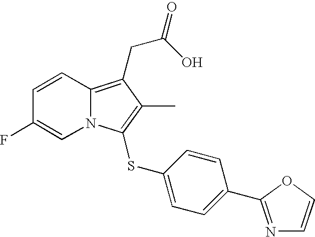

### Preparation 6a

### [6-fluoro-2-methyl-3-(4-oxazol-2-ylphenylsulfanyl)indolizin-1-yl]acetic acid ethyl ester

A mixture of [3-(4-bromophenylsulfanyl)-6-fluoro-2-methylindolizin-1-yl]acetic acid ethyl ester (0.20 g), 2-(tri-n-butylstannyl)oxazole (0.51 g), 1,4-dioxane (3.0 mL) and palladium (0) tetrakis(triphenylphosphine) (0.054 g) was evacuated and flushed with argon several times and then heated at 100° C. for 2 hours. The mixture was cooled to room temperature, concentrated under reduced pressure and the residue partition between dichloromethane and water. The organic phase was washed with water, dried over magnesium sulfate and the solvent removed under reduced pressure. Purification of the residue by column chromatography on silica gel, eluting with a mixture of dichloromethane and methanol (1:0 to 99:1 by volume) gave title compound as a red/brown oil, 0.18 g.

MS: ESI (+ve) (Method B): 411 (M+H)⁺, Retention time 4.5 min.

### Preparation 6b

### [6-fluoro-2-methyl-3-(4-oxazol-2-ylphenylsulfanyl)indolizin-1-yl]acetic acid

The title compound was prepared by the method of Preparation if using [6-fluoro-2-methyl-3-(4-oxazol-2-ylphenylsulfanyl)indolizin-1-yl]acetic acid ethyl ester.

¹H NMR (CDCl₃): δ 2.35 (s, 3H), 3.80 (s, 2H), 6.75 (m, 1H), 6.85 (d, J=8.6 Hz, 2H), 7.15 (s, 1H), 7.35 (dd, J=5.3, 9.7 Hz, 1H), 7.60 (s, 1H), 7.80 (d, J=8.6 Hz, 2H), 8.05 (dd, J=1.8, 4.9 Hz, 1H).

MS: ESI (+ve) (Method A): 383 (M+H)⁺, Retention time 11.4 min.

MS: ESI (+ve) (Method B): 383 (M+H)⁺, Retention time 3.7 min.

### Example 7

### {6-fluoro-2-methyl-3-[4-(1-methyl-1H-imidazol-2-yl)phenylsulfanyl]indolizin-1-yl}acetic acid

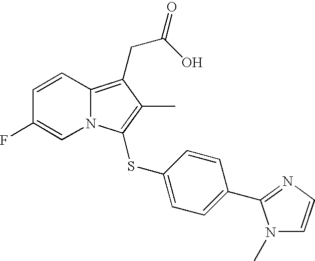

### Preparation 7a

### {6-fluoro-2-methyl-3-[4-(1-methyl-1H-imidazol-2-yl)phenylsulfanyl]indolizin-1-yl}acetic acid ethyl ester

The title compound was prepared by the method of Preparation 6a using [3-(4-bromophenylsulfanyl)-6-fluoro-2-methylindolizin-1-yl]acetic acid ethyl ester and 1-methyl-2-(tri-n-butylstannyl)imidazole.

MS: ESI (+ve) (Method B): 424 (M+H)⁺, Retention time 2.7 min.

### Preparation 7b

### {6-fluoro-2-methyl-3-[4-(1-methyl-1H-imidazol-2-yl)phenylsulfanyl]indolizin-1-yl}acetic acid

The title compound was prepared by the method of Preparation 1f using {6-fluoro-2-methyl-3-[4-(1-methyl-1H-imidazol-2-yl)phenylsulfanyl]indolizin-1-yl}acetic acid ethyl ester.

¹H NMR (CDCl₃): δ 2.35 (s, 3H), 3.55 (s, 3H), 3.75 (s, 2H), 6.70 (m, 1H), 6.85 (d, J=8.3 Hz, 2H), 7.00 (s, 1H), 7.10 (d, J=8.3 Hz, 2H), 7.40 (dd, J=5.4, 9.6 Hz, 1H), 7.05 (s, 1H), 8.05 (dd, J=1.8, 5.1 Hz, 1H).

MS: ESI (+ve) (Method A): 396 (M+H)⁺, Retention time 7.3 min.

### Example 8

### [6-fluoro-2-methyl-3-(4-pyrazol-1-ylphenylsulfanyl)indolizin-1-yl]acetic acid

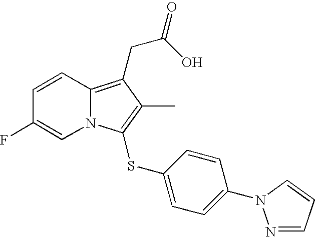

### Preparation 8a

### [6-fluoro-2-methyl-3-(4-pyrazol-1-ylphenylsulfanyl)indolizin-1-yl]acetic acid ethyl ester

A mixture of [3-(4-bromophenylsulfanyl)-6-fluoro-2-methylindolizin-1-yl]acetic acid ethyl ester (0.20 g), pyrazole (0.048 g), cesium carbonate (0.31 g), copper(I) oxide (0.0034 g), pyridine-2-carbaldehyde oxime (0.012 g) and acetonitrile (2.0 mL) was evacuated and flushed with argon several times and then heated at 80° C. for 24 hours. The mixture was diluted with water, extracted with ethyl acetate and the combined extracts washed with water and saturated aqueous sodium chloride solution and then dried over magnesium sulfate. The solvent was removed under reduced pressure and the residue purified by column chromatography on silica gel, eluting with ethyl acetate to afford title compound as a dark red oil, 0.15 g.

MS: ESI (+ve) (Method B): 424 (M+H)⁺, Retention time 2.7 min.

### Preparation 8b

### [6-fluoro-2-methyl-3-(4-pyrazol-1-ylphenylsulfanyl)indolizin-1-yl]acetic acid

The title compound was prepared by the method of Preparation if using [6-fluoro-2-methyl-3-(4-pyrazol-1-ylphenylsulfanyl)indolizin-1-yl]acetic acid ethyl ester.

¹H NMR (CDCl₃): δ 2.35 (s, 3H), 3.75 (s, 2H), 6.40 (t, J=2.0 Hz, 1H), 6.75 (m, 1H), 6.90 (d, J=8.7 Hz, 2H), 7.35 (dd, J=5.2, 9.7 Hz, 1H), 7.50 (d, J=8.7 Hz, 2H), 7.65 (d, J=1.7 Hz, 1H), 7.80 (d, J=2.6 Hz, 1H), 8.05 (m, 1H).

### Example 9 and 10

### {6-cyano-2-methyl-3-[4-(piperazine-1-sulfonyl)phenylsulfanyl]indolizin-1-yl}acetic acid and {7-chloro-6-cyano-2-methyl-3-[4-(piperazine-1-sulfonyl)phenylsulfanyl]indolizin-1-yl}acetic acid

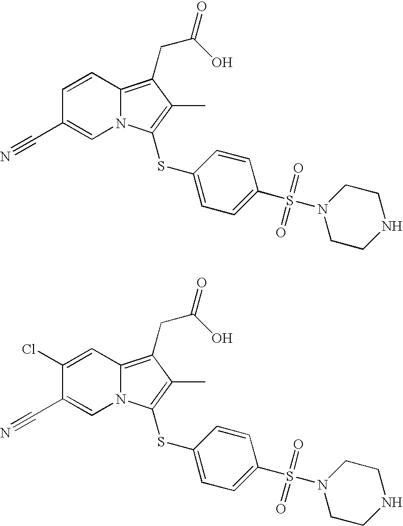

### Preparation 9a and 10a

### bis[4-(4-benzenesulfonyl)piperazine-1-carboxylic acid tert-butyl ester]disulfide

A solution of bis(4-chlorosulfonylphenyl)disulfide (0.50 g) and piperazine-1-carboxylic acid tent-butyl ester (0.67 g) in dichloromethane (20 mL) was treated with N,N-diisopropylethylamine (1.3 mL) and the resulting mixture was stirred at room temperature for 3 hours. The mixture was diluted with dichloromethane, washed with 1.0 M aqueous hydrochloric acid solution, saturated aqueous sodium hydrogen carbonate solution and water and then dried over sodium sulfate. The solvent was removed under the reduced pressure and the residue triturated with diethyl ether to afford title compound as an off-white solid, 0.78 g.

MS: ESI (+ve) (Method B): Retention time 4.7 min.

### Preparation 9b and 10b

### 4-[4-(6-cyano-1-ethoxycarbonylmethyl-2-methylindolizin-3-ylsulfanyl)benzenesulfonyl]piperazine-1-carboxylic acid tert-butyl ester and 4-[4-(7-chloro-6-cyano-1-ethoxycarbonylmethyl-2-methylindolizin-3-ylsulfanyl)benzenesulfonyl]piperazine-1-carboxylic acid tert-butyl ester

The title compounds were prepared by the method of Preparation 1e using (6-cyano-2-methylindolizin-1-yl)acetic acid ethyl ester and bis[4-(4-benzenesulfonyl)piperazine-1-carboxylic acid tert-butyl ester]disulfide.

### Preparation 9c and 10c

### {6-cyano-2-methyl-3-[4-(piperazine-1-sulfonyl)phenylsulfanyl]indolizin-1-yl}acetic acid ethyl ester and {7-chloro-6-cyano-2-methyl-3-[4-(piperazine-1-sulfonyl)phenylsulfanyl]indolizin-1-yl}acetic acid ethyl ester

A mixture of 4-[4-(6-cyano-1-ethoxycarbonylmethyl-2-methylindolizin-3-ylsulfanyl)benzenesulfonyl]piperazine-1-carboxylic acid tert-butyl ester and 4-[4-(7-chloro-6-cyano-1-ethoxycarbonylmethyl-2-methylindolizin-3ylsulfanyl)benzenesulfonyl]piperazine-1-carboxylic acid tert-butyl ester (crude product), dichloromethane (20 mL) and trifluoroacetic acid (5.0 mL) was stirred a room temperature for 3 hours. The mixture was concentrated under reduced pressure and the residue purified by preparative reverse-phase HPLC using a gradient over 30 minutes of acetonitrile in water to afford {6-cyano-2-methyl-3-[4-(piperazine-1-sulfonyl)phenylsulfanyl]indolizin-1-yl}acetic acid ethyl ester as a pale yellow solid (0.039 g) and {7-chloro-6-cyano-2-methyl-3-[4-(piperazine-1-sulfonyl)phenylsulfanyl]indolizin-1-yl}acetic acid ethyl ester as a yellow solid (0.055 g).

### {6-cyano-2-methyl-3-[4-(piperazine-1-sulfonyl)phenylsulfanyl]indolizin-1-yl]acetic acid ethyl ester

MS: ESI (+ve) (Method B): 499 (M+H)⁺, Retention time 2.6 min.

### {7-chloro-6-cyano-2-methyl-3-[4-(piperazine-1-sulfonyl)phenylsulfanyl]indolizin-1-yl}acetic acid ethyl ester

MS: ESI (+ve) (Method B): 533 (M+H)⁺, Retention time 2.7 min.

### Preparation 9d

### {6-cyano-2-methyl-3-[4-(piperazine-1-sulfonyl)phenylsulfanyl]indolizin-1-yl}acetic acid

A solution of {6-cyano-2-methyl-3-[4-(piperazine-1-sulfonyl)phenylsulfanyl]indolizin-1-yl}acetic acid ethyl ester (0.035 g) in tetrahydrofuran (2.0 mL) and water (0.5 mL) was treated with a 1.0 M aqueous lithium hydroxide solution (0.14 mL) and the resulting mixture was stirred at room temperature for 4 hours. The mixture was concentrated under reduced pressure and the residue purified by preparative reverse-phase HPLC using a gradient of acetonitrile in water to afford title compound as a bright yellow solid, 0.022 g.

¹H NMR (DMSO-d6): δ 2.24 (s, 3H), 2.63-2.72 (m, 8H), 3.76 (s, 2H), 7.01 (d, J=8.6 Hz, 2H), 7.05 (dd, J=1.4, 9.3 Hz, 1H), 7.54 (dd, J=8.6 Hz, 2H), 7.69 (d, J=0.9, 9.3 Hz, 1H), 8.92 (m, 1H).

MS: ESI (+ve) (Method A): 471 (M+H)⁺, Retention time 6.9 min.

MS: ESI (+ve) (Method B): 471 (M+H)⁺, Retention time 2.4 min.

### Preparation 10d

### {7-chloro-6-cyano-2-methyl-3-[4-(piperazine-1-sulfonyl)phenylsulfanyl]indolizin-1-yl}acetic acid

A solution of {7-chloro-6-cyano-2-methyl-3-[4-(piperazine-1-sulfonyl)phenylsulfanyl]indolizin-1-yl}acetic acid ethyl ester (0.050 g) in tetrahydrofuran (2.0 mL) and water (0.5 mL) was treated with a 1.0 M aqueous lithium hydroxide solution (0.19 mL) and the resulting mixture was stirred at room temperature for 4 hours. The mixture was concentrated under reduced pressure and the residue purified by preparative reverse-phase HPLC using a gradient of acetonitrile in water to afford title compound as a lemon yellow solid, 0.025 g.

¹H NMR (DMSO-d6): δ 2.21 (s, 3H), 2.71 (m, 8H), 3.78 (s, 2H), 7.03 (d, J=8.5 Hz, 2H), 7.55 (d, J=8.5 Hz, 2H), 8.00 (s, 1H), 9.08 (s, 1H).

MS: ESI (+ve) (Method A): 505 (M+H)⁺, Retention time 7.3 min.

MS: ESI (+ve) (Method B): 505 (M+H)⁺, Retention time 2.5 min.

### Example 11

### {6-cyano-2-methyl-3-[4-(4-methylpiperazine-1-sulfonyl)phenylsulfanyl]indolizin-1-yl}acetic acid

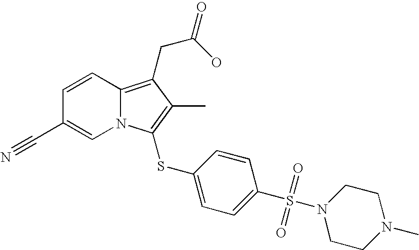

### Preparation 11a

### bis[4-(4-methylpiperazine-1-sulfonyl)benzene]disulfide

A solution of bis(4-chlorosulfonylphenyl)disulfide (0.50 g) and 1-methylpiperazine (0.36 g) in dichloromethane (20 mL) was treated with N,N-diisopropylethylamine (1.3 mL) and the resulting mixture was stirred at room temperature overnight. The mixture was diluted with dichloromethane and extracted with 1.0 M aqueous hydrochloric acid solution. The pH of the aqueous phase was adjusted to 7 by the addition of sodium hydrogen carbonate and then extracted with dichloromethane. The combined extracts were dried over magnesium sulfate and then concentrated under reduced pressure to afford title compound as a white solid, 0.28 g.

MS: ESI (+ve) (Method B): 543 (M+H)⁺, Retention time 2.0 min.

### Preparation 11b

### {6-cyano-2-methyl-3-[4-(4-methylpiperazine-1-sulfonyl)phenylsulfanyl]indolizin-1-yl}acetic acid ethyl ester

The title compounds were prepared by the method of Preparation 1e using (6-cyano-2-methylindolizin-1-yl)acetic acid ethyl ester and bis[4-(4-methylpiperazine-1-sulfonyl)benzene]disulfide.

MS: ESI (+ve) (Method B): 513 (M+H)⁺, Retention time 2.7 min.

### Preparation 11c

### {6-cyano-2-methyl-3-[4-(4-methyl-piperazine-1-sulfonyl)phenylsulfanyl]indolizin-1-yl}acetic acid

A solution of {6-cyano-2-methyl-3-[4-(4-methylpiperazine-1-sulfonyl)phenylsulfanyl]indolizin-1-yl}acetic acid ethyl ester (0.11 g) in tetrahydrofuran (6.0 mL) and water (1.5 mL) was treated with a 1.0 M aqueous lithium hydroxide solution (0.14 mL) and the resulting mixture was stirred at room temperature for 4 hours. The mixture was concentrated under reduced pressure, diluted with water (20 mL) and the pH adjusted to 6 by the addition of 1.0 M aqueous hydrochloric acid solution. The resulting precipitate was collected by filtration and dried to afford title compound as a yellow solid, 0.080 g.

¹H NMR (DMSO-d6): δ 2.07 (s, 3H), 2.24 (s, 3H), 2.27 (m, 4H), 2.79 (m, 4H), 3.77 (s, 2H), 7.00 (d, J=8.6 Hz, 2H), 7.05 (dd, J=1.5, 9.3 Hz, 1H), 7.55 (d, J=8.6 Hz, 2H), 7.69 (dd, J=0.8, 9.3 Hz, 1H), 8.92 (m, 1H).

MS: ESI (+ve) (Method A): 485 (M+H)⁺, Retention time 6.7 min.

MS: ESI (+ve) (Method B): 485 (M+H)⁺, Retention time 2.5 min.

Biological Methods

Compounds of the invention were tested using the following biological test method to determine their ability to displace PGD₂ from the CRTH2 receptor.

Radioligand Binding Assay

The receptor binding assay is performed in a final volume of 200 μL binding buffer [10 mM BES (pH 7.4), 1 mM EDTA, 10 mM manganese chloride, 0.01% BSA] and 1 nM [³H]-PGD₂ (Amersham Biosciences UK Ltd). Ligands are added in assay buffer containing a constant amount of DMSO (1% by volume). Total binding is determined using 1% by volume of DMSO in assay buffer and non-specific binding is determined using 10 μM of unlabeled PGD₂ (Sigma). Human embryonic kidney (HEK) cell membranes (3.5 μg) expressing the CRTH2 receptor are incubated with 1.5 mg wheatgerm agglutinin SPA beads and 1 nM [³H]-PGD₂ (Amersham Biosciences UK Ltd) and the mixture incubated for 3 hours at room temperature. Bound [³H]-PGD₂ is detected using a Microbeta TRILUX liquid scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

Biological Results:

The compounds of the Examples above were tested in the CRTH2 radioligand binding assay described above; the compounds all have IC₅₀ values of less than 1 μM. For example, the compounds of Examples 1 and 4 have Kᵢ values of 0.5 and 19 nM respectively in the CRTH2 radioligand binding assay.

1. A compound of formula (I) or a salt, N-oxide, hydrate, or solvate thereof: 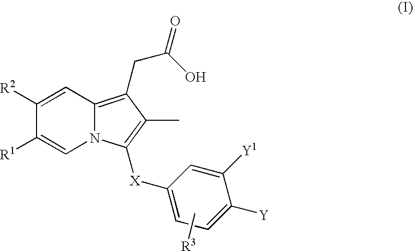
wherein
R¹ is fluoro, chloro, cyano or trifluoromethyl;
R² is hydrogen, fluoro or chloro;
R³ is hydrogen, fluoro, chloro or trifluoromethyl;
X is —CH₂—, —S—, —S(═O)— or —S(═O)₂—;
one of Y and Y¹ is hydrogen and the other is —C(═O)R⁴, —S(═O)₂R⁴, —CR⁵R⁶OR⁷, or a heterocyclic group selected from furanyl, thienyl, pyrrolyl, oxazolyl, thiazolyl, imidazolyl, pyrazolyl, isoxazolyl, isothiazolyl, 1,2,3-oxadiazolyl, 1,2,4-oxadiazolyl, 1,3,4-oxadiazolyl, 1,2,5-oxadiazolyl, furazanyl, 1,2,4-triazolyl, 1,2,3-thiadiazolyl, 1,2,5-thiadiazolyl, 1,3,4-thiadiazolyl, 1,2,4-thiadiazolyl, tetrazolyl, pyridinyl, pyridazinyl, pyrimidinyl, pyrazinyl, 1,2,4-triazinyl and 1,3,5-triazinyl, any of which may be optionally substituted;
R⁴ is an optionally substituted cyclic amino group having 5, 6 or 7 ring atoms which is linked to the carbonyl or sulfonyl through a ring nitrogen;
R⁵ and R⁶ are independently hydrogen, (C₁-C₃)alkyl, or cyclopropyl, or R⁵ and R⁶ taken together with the carbon atom to which they are attached form a 3-6 membered cycloalkyl ring; and
R⁷ is optionally substituted (C₁-C₆)alkyl or (C₃-C₆)cycloalkyl.

2. The compound as claimed in claim 1 wherein X is —CH₂— or —S—.

3. The compound as claimed in claim 1 wherein Y or Y¹ is any of the heterocyclic groups defined therein and optional substituents therein are selected from halogen, —CN, C₁-C₃alkyl, fully or partially fluorinated C₁-C₃alkyl, and cyclopropyl.

4. The compound as claimed in claim 1 wherein Y or Y¹ is —S(═O)₂R⁴ wherein R⁴ is morpholinyl, piperidinyl, piperazinyl, 4-methylpiperazinyl, or pyrrolidinyl.

5. The compound as claimed in claim 1 wherein Y or Y¹ is —CR⁵R⁶OR⁷ wherein R⁵ and R⁶ are independently hydrogen or methyl, and R⁷ is methyl or ethyl.

6. The compound as claimed in claim 1 wherein Y¹ is hydrogen.

7. (canceled)

8. A pharmaceutical composition comprising a compound as claimed in claim 1, and a pharmaceutically acceptable carrier.

9-10. (canceled)

11. A method of treatment of asthma, chronic obstructive pulmonary disease, rhinitis, allergic airway syndrome, or allergic rhinobronchitis, comprising administering to a patient suffering such disease an effective amount of a compound as claimed in claim 1.

12. A method of treatment of psoriasis, atopic or non-atopic dermatitis, Crohn's disease, ulcerative colitis, or irritable bowel disease, comprising administering to a patient suffering such disease an effective amount of a compound as claimed in claim 1.
